(19) United States
(12) Patent Application Publication
(10) Pub. No.: US 2005/0244953 A1
(43) Pub. Date: Nov. 3, 2005

(54) Techniques for controlling the optical properties of assay devices

(75) Inventor: David Samuel Cohen, Alpharetta, GA (US)

Correspondence Address: DORITY & MANNING, P.A., POST OFFICE BOX 1449, GREENVILLE, SC, 29602-1449, US

(73) Assignee: Kimberly-Clark Worldwide, Inc.

(21) Appl. No.: 11/022,286

(22) Filed: Dec. 22, 2004

Related U.S. Application Data
(60) Provisional application No. 60/608,941, filed on Apr. 30, 2004.

(51) Int. Cl. C12M 1/34; C12Q 1/68

(52) U.S. Cl. 435/287.2

(57) ABSTRACT

A system that employs optical detection techniques to identify the presence or quantity of an analyte residing in a test sample is provided. Unlike conventional systems, the optical detection system of the present invention uses the assay device itself to enhance the ability of an optical reader to detect the presence or absence of the analyte. In particular, the support for the assay device is provided with one or more of the optical properties desired for the optical reader to enhance its operation. This allows for the use of optical readers that are relatively simple, portable, and inexpensive.

### RELATED APPLICATIONS

The present application claims priority to a provisional application having Ser. No. 60/608,941, which was filed on Mar. 30, 2004.

### BACKGROUND OF THE INVENTION

Optical detection techniques (e.g., fluorescence, phosphorescence, reflectance, diffraction, and so forth) have been employed to quantitatively determine the presence or absence of an analyte. For example, conventional fluorescence readers utilize an excitation source that causes fluorescent labels to emit photons at a certain wavelength. A detector registers the emission photons and produces a recordable output, usually as an electrical signal or a photographic image. In addition, the readers often utilize one or more optical elements to help focus, shape, or attenuate the transmitted fluorescent signals in a desired manner. For example, optical filters are sometimes utilized to isolate the emission photons from the excitation photons.

However, one problem with conventional optical optical detection systems is that they utilize very complex optical elements, and thus are often bulky, non-portable, and expensive. In addition, some conventional optical detection systems are also problematic when used in conjunction with assay devices that contain a chromatographic medium, such as a porous membrane. For example, in a membrane-based device, the concentration of the analyte is reduced because it is diluted by a liquid that can flow through the porous membrane. Unfortunately, background interference becomes increasingly problematic at such low analyte concentrations because the intensity to be detected is relatively low. Because the structure of the membrane also tends to reflect and/or diffuse the emitted light, the ability of the detector to accurately measure the intensity of the labeled analyte is substantially reduced. In fact, the intensity of the signal is typically three to four orders of magnitude smaller than the excitation light reflected by the porous membrane.

As such, a need currently exists for an improved technique for quantitatively determining the presence or absence of an analyte within a test sample. In particular, a need exists for a simple, inexpensive, and effective optical detection system that utilizes a chromatographic-based assay device.

### SUMMARY OF THE INVENTION

In accordance with one embodiment of the present invention, an optical detection system for detecting the presence or quantity of an analyte residing in a test sample is disclosed. The system comprises an optical reader that comprises an illumination source and a detector, the illumination source being capable of providing electromagnetic radiation and the detector being capable of registering a detection signal. The system further comprises an assay device that includes a porous membrane having a first surface and an opposing second surface. The porous membrane is in communication with detection probes that are capable of producing the detection signal when contacted with the electromagnetic radiation. The first surface of the porous membrane is carried by a support, the support having a thickness of from about 100 to about 5,000 micrometers. The support is also provided with an optically functional material that is selectively tailored to one or more optical properties of the optical reader.

In accordance with another embodiment of the present invention, an optical detection system for detecting the presence or quantity of an analyte residing in a test sample is disclosed. The system comprises an optical reader that comprises an illumination source and a detector, the illumination source being capable of providing electromagnetic radiation and the detector being capable of registering a detection signal. The system further comprises an assay device that includes a porous membrane having a first surface and an opposing second surface. The porous membrane is in communication with detection probes that are capable of producing the detection signal when contacted with the electromagnetic radiation. The illumination source and detector are positioned on opposing sides of the assay device so that the porous membrane is positioned in the electromagnetic radiation path defined between the illumination source and detector. In addition, the first surface of the porous membrane is carried by an optically transmissive support, the optically transmissive support having a thickness of from about 150 to about 2,000 micrometers and being provided with an optically functional material that is selectively tailored to one or more optical properties of the optical reader.

In accordance with still another embodiment of the present invention, a method for detecting the presence or quantity of an analyte within a test sample is disclosed. An optical reader and a chromatographic medium for an assay device are provided. The method comprises selectively controlling the optical properties of a support for the chromatographic medium to correspond with one or more optical requirements of the optical reader. The support has a thickness of from about 100 to about 5,000 micrometers. In some embodiments, the method further comprises contacting the test sample with the chromatographic medium; supplying electromagnetic radiation to the test sample to cause the production of a detection signal; and registering the detection signal.

Other features and aspects of the present invention are discussed in greater detail below.

### BRIEF DESCRIPTION OF THE DRAWINGS

A full and enabling disclosure of the present invention, including the best mode thereof, directed to one of ordinary skill in the art, is set forth more particularly in the remainder of the specification, which makes reference to the appended figure in which:

FIG. 1 is a perspective view of one embodiment of an optical detection system that may be used in the present invention; and

FIG. 2 schematically illustrates various embodiments of the optical detection system, in which FIG. 2a illustrates an embodiment in which the illumination source and detector are spaced relatively distant from the assay device; FIG. 2b illustrates the embodiment of FIG. 2a in which an illumination lens and a detection lens are also used to focus light to and from the assay device; FIG. 2c illustrates the embodiment of FIG. 2b in which the illumination lens is removed and the illumination source is moved closer to the assay device; and FIG. 2d illustrates the embodiment of FIG. 2c in which the detection lens is removed and the detector is moved closer to the assay device.

Repeat use of reference characters in the present specification and drawings is intended to represent same or analogous features or elements of the invention.

### DETAILED DESCRIPTION OF REPRESENTATIVE EMBODIMENTS

### Definitions

As used herein, the term “analyte” generally refers to a substance to be detected. For instance, analytes may include antigenic substances, haptens, antibodies, and combinations thereof. Analytes include, but are not limited to, toxins, organic compounds, proteins, peptides, microorganisms, amino acids, nucleic acids, hormones, steroids, vitamins, drugs (including those administered for therapeutic purposes as well as those administered for illicit purposes), drug intermediaries or byproducts, bacteria, virus particles and metabolites of or antibodies to any of the above substances. Specific examples of some analytes include ferritin; creatinine kinase MB (CK-MB); digoxin; phenytoin; phenobarbitol; carbamazepine; vancomycin; gentamycin; theophylline; valproic acid; quinidine; luteinizing hormone (LH); follicle stimulating hormone (FSH); estradiol, progesterone; C-reactive protein; lipocalins; IgE antibodies; cytokines; vitamin B2 micro-globulin; glycated hemoglobin (Gly. Hb); cortisol; digitoxin; N-acetylprocainamide (NAPA); procainamide; antibodies to rubella, such as rubella-IgG and rubella IgM; antibodies to toxoplasmosis, such as toxoplasmosis IgG (Toxo-IgG) and toxoplasmosis IgM (Toxo-IgM); testosterone; salicylates; acetaminophen; hepatitis B virus surface antigen (HBsAg); antibodies to hepatitis B core antigen, such as anti-hepatitis B core antigen IgG and IgM (Anti-HBC); human immune deficiency virus 1 and 2 (HIV 1 and 2); human T-cell leukemia virus 1 and 2 (HTLV); hepatitis B e antigen (HBeAg); antibodies to hepatitis B e antigen (Anti-HBe); influenza virus; thyroid stimulating hormone (TSH); thyroxine (T4); total triiodothyronine (Total T3); free triiodothyronine (Free T3); carcinoembryoic antigen (CEA); lipoproteins, cholesterol, and triglycerides; and alpha fetoprotein (AFP). Drugs of abuse and controlled substances include, but are not intended to be limited to, amphetamine; methamphetamine; barbiturates, such as amobarbital, secobarbital, pentobarbital, phenobarbital, and barbital; benzodiazepines, such as librium and valium; cannabinoids, such as hashish and marijuana; cocaine; fentanyl; LSD; methaqualone; opiates, such as heroin, morphine, codeine, hydromorphone, hydrocodone, methadone, oxycodone, oxymorphone and opium; phencyclidine; and propoxyhene. Other potential analytes may be described in U.S. Pat. No. 6,436,651 to Everhart, et al. and U.S. Pat. No. 4,366,241 to Tom et al.

As used herein, the term “test sample” generally refers to a biological material suspected of containing the analyte. The test sample may be derived from any biological source, such as a physiological fluid, including, blood, interstitial fluid, saliva, ocular lens fluid, cerebral spinal fluid, sweat, urine, milk, ascites fluid, mucous, nasal fluid, sputum, synovial fluid, peritoneal fluid, vaginal fluid, menses, amniotic fluid, semen, and so forth. Besides physiological fluids, other liquid samples may be used such as water, food products, and so forth, for the performance of environmental or food production assays. In addition, a solid material suspected of containing the analyte may be used as the test sample. The test sample may be used directly as obtained from the biological source or following a pretreatment to modify the character of the sample. For example, such pretreatment may include preparing plasma from blood, diluting viscous fluids, and so forth. Methods of pretreatment may also involve filtration, precipitation, dilution, distillation, mixing, concentration, inactivation of interfering components, the addition of reagents, lysing, etc. Moreover, it may also be beneficial to modify a solid test sample to form a liquid medium or to release the analyte.

### Detailed Description

Reference now will be made in detail to various embodiments of the invention, one or more examples of which are set forth below. Each example is provided by way of explanation of the invention, not limitation of the invention. In fact, it will be apparent to those skilled in the art that various modifications and variations may be made in the present invention without departing from the scope or spirit of the invention. For instance, features illustrated or described as part of one embodiment, may be used on another embodiment to yield a still further embodiment. Thus, it is intended that the present invention covers such modifications and variations as come within the scope of the appended claims and their equivalents.

In general, the present invention is directed to a system that employs optical detection techniques to identify the presence or quantity of an analyte residing in a test sample. Unlike conventional systems, the optical detection system of the present invention uses the assay device itself to enhance the ability of an optical reader to detect the presence or absence of the analyte. In particular, the support for the assay device is provided with one or more of the optical properties desired for the optical reader to enhance its operation. This allows for the use of optical readers that are relatively simple, portable, and inexpensive.

The assay device of the present invention generally contains a chromatographic medium carried by a support. The chromatographic medium may be made from any of a variety of materials through which the test sample is capable of passing, such as a fluidic channel, porous membrane, etc. Likewise, the medium may be made from a material through which electromagnetic radiation may transmit, such as an optically diffuse (e.g., translucent) or transparent material. For example, the chromatographic medium may be a porous membrane formed from materials such as, but not limited to, natural, synthetic, or naturally occurring materials that are synthetically modified, such as polysaccharides (e.g., cellulose materials such as paper and cellulose derivatives, such as cellulose acetate and nitrocellulose); polyether sulfone; polyethylene; nylon; polyvinylidene fluoride (PVDF); polyester; polypropylene; silica; inorganic materials, such as deactivated alumina, diatomaceous earth, MgSO₄, or other inorganic finely divided material uniformly dispersed in a porous polymer matrix, with polymers such as vinyl chloride, vinyl chloride-propylene copolymer, and vinyl chloride-vinyl acetate copolymer; cloth, both naturally occurring (e.g., cotton) and synthetic (e.g., nylon or rayon); porous gels, such as silica gel, agarose, dextran, and gelatin; polymeric films, such as polyacrylamide; and so forth. In one particular embodiment, the chromatographic medium is formed from nitrocellulose and/or polyether sulfone materials. It should be understood that the term “nitrocellulose” refers to nitric acid esters of cellulose, which may be nitrocellulose alone, or a mixed ester of nitric acid and other acids, such as aliphatic carboxylic acids having from 1 to 7 carbon atoms.

The size and shape of the chromatographic medium may generally vary as is readily recognized by those skilled in the art. For instance, a porous membrane strip may have a length of from about 10 to about 100 millimeters, in some embodiments from about 20 to about 80 millimeters, and in some embodiments, from about 40 to about 60 millimeters. The width of the membrane strip may also range from about 0.5 to about 20 millimeters, in some embodiments from about 1 to about 15 millimeters, and in some embodiments, from about 2 to about 10 millimeters. Likewise, the thickness of the membrane strip is generally small enough to allow transmission-based detection. For example, the membrane strip may have a thickness less than about 500 micrometers, in some embodiments less than about 250 micrometers, and in some embodiments, less than about 150 micrometers. For instance, one suitable membrane strip having a thickness of about 125 micrometers may be obtained from Millipore Corp. of Bedford, Mass. under the name “SHF180UB25.”

As stated above, the support carries the chromatographic medium. For example, the support may be positioned directly adjacent to the chromatographic medium, or one or more intervening layers may be positioned between the chromatographic medium and the support. Regardless, the support may generally be formed from any material able to carry the chromatographic medium. Although not required, the support is typically optically transmissive (e.g., transparent, optically diffuse, etc.) so that light passes therethrough. In addition, it is also generally desired that the support is liquid-impermeable so that fluid flowing through the medium does not leak through the support. Examples of suitable materials for the support include, but are not limited to, glass; polymeric materials, such as polystyrene, polypropylene, polyester (e.g., Mylar® film), polybutadiene, polyvinylchloride, polyamide, polycarbonate, epoxides, methacrylates, and polymelamine; and so forth. To provide a sufficient structural backing for the chromatographic medium, the support is generally selected to have a certain minimum thickness. Likewise, the thickness of the support is typically not so larger as to adversely affect its optical properties. Thus, for example, the support may have a thickness that ranges from about 100 to about 5,000 micrometers, in some embodiments from about 150 to about 2,000 micrometers, and in some embodiments, from about 250 to about 1,000 micrometers.

As is well known the art, the chromatographic medium may be cast onto the support, wherein the resulting laminate may be die-cut to the desired size and shape. Alternatively, the chromatographic medium may be laminated to the support with an adhesive. In some embodiments, a nitrocellulose or nylon porous membrane is adhered to a Mylar® film. An adhesive is used to bind the porous membrane to the Mylar® film, such as a pressure-sensitive adhesive. Laminate structures of this type are believed to be commercially available from Millipore Corp. of Bedford, Mass. Still other examples of suitable laminate assay device structures are described in U.S. Pat. No. 5,075,077 to Durley, III, et al., which is incorporated herein in its entirety by reference thereto for all purposes.

The selection of an adhesive for laminating the support, the chromatographic medium, and/or any other layer of the device may depend on a variety of factors, including the desired optical properties of the detection system and the materials used to form the assay device. For example, in some embodiments, the selected adhesive is optically transparent and compatible with the porous membrane and support. Optical transparency may minimize any adverse affect that the adhesive might otherwise have on the optical detection system. Suitable optically transparent adhesives may be formed, for instance, from acrylate or (meth)acrylate polymers, such as polymers of (meth)acrylate esters, acrylic or (meth)acrylic acid monomers, and so forth. Exemplary (meth)acrylate ester monomers include monofunctional acrylate or methacrylate esters of non-tertiary alkyl alcohols, such as methyl acrylate, ethyl acrylate, propyl acrylate, n-butyl acrylate, isobutyl acrylate, 2-methylbutyl acrylate, 2-ethylhexyl acrylate, 2-ethylhexyl methacrylate, n-octyl acrylate, n-octyl methacrylate, isooctyl acrylate, isooctyl methacrylate, isononyl acrylate, isodecyl acrylate, isobomyl acrylate, isobornyl methacrylate, vinyl acetate, and mixtures thereof. Exemplary (meth)acrylic acid monomers include acrylic acid, methacrylic acid, beta-carboxyethyl acrylate, itaconic acid, crotonic acid, fumaric acid, and so forth. Several examples of such optically transparent adhesives are described in U.S. Pat. No. 6,759,121 to Alahapperuma. et al., which is incorporated herein in its entirety by reference thereto for all purposes. Further, suitable transparent adhesives may also be obtained from Adhesives Research, Inc. of Glen Rock, Pa. under the name ARclear® 8154, which is an unsupported optically clear acrylic pressure-sensitive adhesive. Other suitable transparent adhesives may be obtained from 3M Corp. of St. Paul, Minn. under the names “9843” or “8146.” In addition, the manner in which the adhesive is applied may also enhance the optical properties of the assay device. For instance, the adhesive may enhance certain optical properties of the support (e.g., diffusiveness). Thus, in one particular embodiment, such an adhesive may be applied in a pattern that corresponds to the areas in which enhanced optical properties are desired.

Regardless of the manner in which it is constructed, the present inventor has discovered that the optical properties of the assay device may be selectively controlled to enhance the overall efficiency and effectiveness of the assay device. Specifically, one or more optical properties of the support, e.g., reflectivity, transmittance, refractivity, polarization, absorbance, etc., to the requirements of the optical reader. The support may, for example, attenuate one or more wavelengths of light. The attenuation of the light may involve extinction or enhancement of specific wavelengths of light as in an antireflective optical stack for a visually observable color change, or may involve modifying the intensity of a specific wavelength of light upon reflection or transmittance. The support may also modify the optical parameters to allow a change in the state or degree of polarization in the incident light.

The support may be optimized for a particular optical property in a variety of ways. For example, the material(s) used for forming the support may simply be selected to possess the desired optical property. Alternatively, an optically functional material may be applied to the support before and/or after forming the assay device. Such an optically functional material may be applied to the support in a variety of ways. For example, the optically functional material may be dyed or coated onto one or more surfaces of the support. When applied in this manner, the optically functional material may cover only a portion or an entire surface of the support. In one embodiment, for example, the optically functional material is applied to a portion of the support that corresponds to the detection zone or calibration zones of the device. In this manner, the optically functional material may enhance the detection or calibration signals produced by the assay device during use. Alternatively, the optically functional material may also be incorporated into the structure of the support. For example, internal optics may be formed using known techniques, such as embossing, stamping, molding, etc.

The support is generally optimized for the desired application of the assay device and for the method of analysis used to interpret the results. For example, the support may contain an optical filter, e.g., high-pass (allows only high frequencies to pass), low-pass (allows only low frequencies to pass), or bandpass (allows only a limited range of frequencies to pass), which optimizes the operability of an excitation source or detector. Several examples of suitable optical filters include, but are not limited to, dyed plastic resin or gelatin filters, dichroic filters, thin multi-layer film interference filters, plastic or glass filters, epoxy or cured transparent resin filters, and so forth. In other embodiments, the support may contain a mask that prevents light from passing through one or more sections thereof, such as a black coating or dye. The support may also focus, shape, and/or direct light into a form that is optimal for subsequent detection. For example, light guiding elements may direct light in a desired direction, such as a single optical fiber, fiber bundle, segment of a bifurcated fiber bundle, large diameter light pipe, planar waveguide, attenuated total reflectance crystal, dichroic mirror, plane mirror or other light guiding elements.

In addition, a lens may also be used to collect and focus light. One particular embodiment of the present invention utilizes a micro-lens (e.g., having a size less than about 2 millimeters and arranged in two or more dimensions) to focus light toward the test sample and/or optical detector. Suitable micro-optic lenses include, but are not limited to, gradient index (GRIN) lenses, ball lenses, Fresnel lenses, and so forth. For example, a gradient index lens is generally cylindrical, and has a refractive index that changes radially with a parabolic profile. A ball lens is generally spherical, and has a refractive index that is radially constant. Because of their relatively small size, such lenses may be particularly advantageous in the present invention. Still other examples of suitable optically functional materials are described in U.S. Pat. No. 5,827,748 to Golden; U.S. Pat. No. 6,084,683 to Bruno, et al.; U.S. Pat. No. 6,556,299 to Rushbrooke, et al.; and U.S. Pat. No. 6,566,508 to Bentsen, et al., which are incorporated herein in their entirety by reference thereto for all purposes. Any of a variety of well-known techniques may be utilized to form such a micro-lens. For example, micro-lenses may be formed by submerging a substrate (e.g., silicon or quartz) into a solution of alkaline salt so that ions are exchanged between the substrate and the salt solution through a mask formed on the substrate, thereby obtaining a substrate having a distribution of indexes of refraction corresponding to the pattern of the mask. In addition, a photosensitive monomer may be irradiated with ultraviolet rays to polymerize an irradiated portion of the photosensitive monomer. Thus, the irradiated portion bulges into a lens configuration under an osmotic pressure occurring between the irradiated portion and the non-irradiated portion. In another embodiment, a photosensitive resin may be patterned into circles, and heated to temperatures above its softening point to enable the peripheral portion of each circular pattern to sag by surface tension. This process is referred to as a “heat sagging process.” Further, a lens substrate may simply be mechanically shaped into a lens. Still other suitable techniques for forming a micro-lens or other micro-optics are described in U.S. Pat. No. 5,225,935 to Watanabe, et al.; U.S. Pat. No. 5,910,940 to Guerra; and U.S. Pat. No. 6,411,439 to Nishikawa, which are incorporated herein in their entirety by reference thereto for all purposes.

Optical diffusers may also be utilized that scatter light in a certain direction. Optical diffusers are particularly useful in conjunction with a detection system that employs a “point” light source, such as a light-emitting diode (LED). For example, suitable optical diffusers may include diffusers that scatter light in various directions, such as ground glass, opal glass, opaque plastics, chemically etched plastics, machined plastics, and so forth. Opal glass diffusers contain a milky white “opal” coating for evenly diffusing light, thereby producing a near Lambertian source. Other suitable light-scattering diffusers include polymeric materials (e.g., polyesters, polycarbonates, etc.) that contain a light-scattering material, such as titanium dioxide or barium sulfate particles. In other embodiments, holographic diffusers may be utilized that both homogenizes and imparts predetermined directionality to light rays emanating from a light source. Such diffusers may contain a micro-sculpted surface structure that controls the direction in which light propagates in either reflection or transmission. Examples of such holographic diffusers are described in more detail in U.S. Pat. No. 5,534,386 to Petersen, et al., which is incorporated herein in its entirety by reference thereto for all purposes. Still other examples of optically functional materials that may be used in the present invention described in U.S. Pat. No. 5,827,748 to Golden; U.S. Pat. No. 6,084,683 to Bruno, et al.; U.S. Pat. No. 6,556,299 to Rushbrooke, et al.; and U.S. Pat. No. 6,566,508 to Bentsen, et al., which are incorporated herein in their entirety by reference thereto for all purposes.

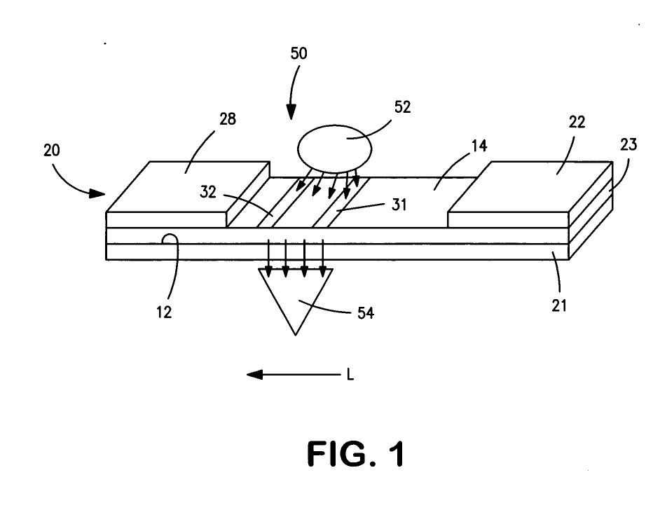

Referring to FIG. 1, one embodiment of an optical detection system in which the optically functional support of the present invention may be incorporated will now be described in more detail. As shown, the optical detection system contains an assay device 20, which includes a chromatographic medium 23 having a first surface 12 and an opposing second surface 14. The first surface 12 of the medium 23 is positioned adjacent to a support 21. An absorbent pad 28 is provided on the second surface 14 that generally receives fluid after it migrates through the entire chromatographic medium 23. As is well known in the art, the absorbent pad 28 may also assist in promoting capillary action and fluid flow through the chromatographic medium 23. To initiate the detection of an analyte within the test sample, a user may directly apply the test sample to a portion of the chromatographic medium 23 through which it may then travel in the direction illustrated by arrow “L” in FIG. 1. Alternatively, the test sample may first be applied to a sample pad (not shown) that is in fluid communication with the chromatographic medium 23. Some suitable materials that may be used to form the absorbent pad 28 and/or sample pad include, but are not limited to, nitrocellulose, cellulose, porous polyethylene pads, and glass fiber filter paper. If desired, the sample pad may also contain one or more assay pretreatment reagents, either diffusively or non-diffusively attached thereto.

In the illustrated embodiment, the test sample travels from the sample pad (not shown) to a conjugate pad 22 that is placed in communication with one end of the sample pad. The conjugate pad 22 is formed from a material through which a fluid is capable of passing. For example, in one embodiment, the conjugate pad 22 is formed from glass fibers. Although only one conjugate pad 22 is shown, it should be understood that other conjugate pads may also be used in the present invention.

To facilitate accurate detection of the presence or absence of an analyte within the test sample, a predetermined amount of detection probes may applied at one or more locations of the assay device 20, such as to the conjugate pad 22. Any substance generally capable of generating a signal that is detectable visually or by an instrumental device may be used as detection probes. Various suitable substances may include chromogens; luminescent compounds (e.g., fluorescent, phosphorescent, etc.); radioactive compounds; visual labels (e.g., latex particles or colloidal metallic particles, such as gold); liposomes or other vesicles containing signal producing substances; and so forth. Other suitable detectable substances may be described in U.S. Pat. No. 5,670,381 to Jou, et al. and U.S. Pat. No. 5,252,459 to Tarcha, et al., which are incorporated herein in their entirety by reference thereto for all purposes.

In some embodiments, the detection probes may contain a luminescent compound that produces an optically detectable signal. The luminescent compound may be a molecule, polymer, dendrimer, particle, and so forth. For example, suitable fluorescent molecules may include, but not limited to, fluorescein, europium chelates, phycobiliprotein, rhodamine, and their derivatives and analogs. Other suitable fluorescent compounds are semiconductor nanocrystals commonly referred to as “quantum dots.” For example, such nanocrystals may contain a core of the formula CdX, wherein X is Se, Te, S, and so forth. The nanocrystals may also be passivated with an overlying shell of the formula YZ, wherein Y is Cd or Zn, and Z is S or Se. Other examples of suitable semiconductor nanocrystals may also be described in U.S. Pat. No. 6,261,779 to Barbera-Guillem, et al. and U.S. Pat. No. 6,585,939 to Dapprich, which are incorporated herein in their entirety by reference thereto for all purposes.

Further, suitable phosphorescent compounds may include metal complexes of one or more metals, such as ruthenium, osmium, rhenium, iridium, rhodium, platinum, indium, palladium, molybdenum, technetium, copper, iron, chromium, tungsten, zinc, and so forth. Especially preferred are ruthenium, rhenium, osmium, platinum, and palladium. The metal complex may contain one or more ligands that facilitate the solubility of the complex in an aqueous or nonaqueous environment. For example, some suitable examples of ligands include, but are not limited to, pyridine; pyrazine; isonicotinamide; imidazole; bipyridine; terpyridine; phenanthroline; dipyridophenazine; porphyrin, porphine, and derivatives thereof. Such ligands may be, for instance, substituted with alkyl, substituted alkyl, aryl, substituted aryl, aralkyl, substituted aralkyl, carboxylate, carboxaldehyde, carboxamide, cyano, amino, hydroxy, imino, hydroxycarbonyl, aminocarbonyl, amidine, guanidinium, ureide, sulfur-containing groups, phosphorus containing groups, and the carboxylate ester of N-hydroxy-succinimide.

Porphyrins and porphine metal complexes possess pyrrole groups coupled together with methylene bridges to form cyclic structures with metal chelating inner cavities. Many of these molecules exhibit strong phosphorescence properties at room temperature in suitable solvents (e.g., water) and an oxygen-free environment. Some suitable porphyrin complexes that are capable of exhibiting phosphorescent properties include, but are not limited to, platinum (II) coproporphyrin-I and IIII, palladium (II) coproporphyrin, ruthenium coproporphyrin, zinc(II)-coproporphyrin-I, derivatives thereof, and so forth. Similarly, some suitable porphine complexes that are capable of exhibiting phosphorescent properties include, but not limited to, platinum(II) tetra-meso-fluorophenylporphine and palladium(II) tetra-meso-fluorophenylporphine. Still other suitable porphyrin and/or porphine complexes are described in U.S. Pat. No. 4,614,723 to Schmidt, et al.; U.S. Pat. No. 5,464,741 to Hendrix; U.S. Pat. No. 5,518,883 to Soini; U.S. Pat. No. 5,922,537 to Ewart, et al.; U.S. Pat. No. 6,004,530 to Sagner, et al.; and U.S. Pat. No. 6,582,930 to Ponomarev, et al., which are incorporated herein in their entirety by reference thereto for all purposes.

Bipyridine metal complexes may also be utilized as phosphorescent compounds. Some examples of suitable bipyridine complexes include, but are note limited to, bis[(4,4′-carbomethoxy)-2,2′-bipyridine] 2-[3-(4-methyl-2,2′-bipyridine-4-yl)propyl]-1,3-dioxolane ruthenium (II); bis(2,2′bipyridine)[4-(butan-1-al)-4′-methyl-2,2′-bi-pyridine]ruthenium (II); bis(2,2′-bipyridine)[4-(4′-methyl-2,2′-bipyridine-4′-yl)-butyric acid] ruthenium (II); tris(2,2′bipyridine)ruthenium (II); (2,2′-bipyridine) [bis-bis(1,2-diphenylphosphino)ethylene] 2-[3-(4-methyl-2,2′-bipyridine -4′-yl)propyl]-1,3-dioxolane osmium (II); bis(2,2′-bipyridine)[4-(4′-methyl-2,2′-bipyridine)-butylamine]ruthenium (II); bis(2,2′-bipyridine)[1-bromo-4(4′-methyl-2,2′-bipyridine-4-yl)butane]ruthenium (II); bis(2,2′-bipyridine)maleimidohexanoic acid, 4-methyl-2,2′-bipyridine-4′-butylamide ruthenium (II), and so forth. Still other suitable metal complexes that may exhibit phosphorescent properties may be described in U.S. Pat. No. 6,613,583 to Richter, et al.; U.S. Pat. No. 6,468,741 to Massey, et al.; U.S. Pat. No. 6,444,423 to Meade, et al.; U.S. Pat. No. 6,362,011 to Massey, et al.; U.S. Pat. No. 5,731,147 to Bard, et al.; and U.S. Pat. No. 5,591,581 to Massey, et al., which are incorporated herein in their entirety by reference thereto for all purposes.

“Time-resolved” luminescent detection techniques may be utilized in some embodiments of the present invention. Time-resolved detection involves exciting a luminescent probe with one or more short pulses of light, then typically waiting a certain time after excitation before measuring the remaining luminescent signal, such as from about 1 to about 200 microseconds, and particularly from about 10 to about 50 microseconds. In this manner, any short-lived phosphorescent or fluorescent background signals and scattered excitation radiation are eliminated. This ability to eliminate much of the background signals may result in sensitivities that are 2 to 4 orders greater than conventional fluorescence or phosphorescence. Thus, time-resolved detection is designed to reduce background signals from the illumination source or from scattering processes (resulting from scattering of the excitation radiation) by taking advantage of the characteristics of certain luminescent materials.

To function effectively, time-resolved techniques generally require a relatively long emission lifetime for the luminescent compounds. This is desired so that the compound emits its signal well after any short-lived background signals dissipate. Furthermore, a long luminescence lifetime makes it possible to use low-cost circuitry for time-gated measurements. For example, the detectable compounds may have a luminescence lifetime of greater than about 1 microsecond, in some embodiments greater than about 10 microseconds, in some embodiments greater than about 50 microseconds, and in some embodiments, from about 100 microseconds to about 1000 microseconds. In addition, the compound may also have a relatively large “Stokes shift.” The term “Stokes shift” is generally defined as the displacement of spectral lines or bands of luminescent radiation to a longer emission wavelength than the excitation lines or bands. A relatively large Stokes shift allows the excitation wavelength of a luminescent compound to remain far apart from its emission wavelengths and is desirable because a large difference between excitation and emission wavelengths makes it easier to eliminate the reflected excitation radiation from the emitted signal. Further, a large Stokes shift also minimizes interference from luminescent molecules in the sample and/or light scattering due to proteins or colloids, which are present with some body fluids (e.g., blood). In addition, a large Stokes shift also minimizes the requirement for expensive, high-precision filters to eliminate background interference. For example, in some embodiments, the luminescent compounds have a Stokes shift of greater than about 50 nanometers, in some embodiments greater than about 100 nanometers, and in some embodiments, from about 100 to about 350 nanometers.

For example, one suitable type of fluorescent compound for use in time-resolved detection techniques includes lanthanide chelates of samarium (Sm (III)), dysprosium (Dy (III)), europium (Eu (III)), and terbium (Tb (III)). Such chelates may exhibit strongly red-shifted, narrow-band, long-lived emission after excitation of the chelate at substantially shorter wavelengths. Typically, the chelate possesses a strong ultraviolet excitation band due to a chromophore located close to the lanthanide in the molecule. Subsequent to excitation by the chromophore, the excitation energy may be transferred from the excited chromophore to the lanthanide. This is followed by a fluorescence emission characteristic of the lanthanide. Europium chelates, for instance, have exceptionally large Stokes shifts of about 250 to about 350 nanometers, as compared to only about 28 nanometers for fluorescein. Also, the fluorescence of europium chelates is long-lived, with lifetimes of about 100 to about 1000 microseconds, as compared to about 1 to about 100 nanoseconds for other fluorescent labels. In addition, these chelates have a narrow emission spectra, typically having bandwidths less than about 10 nanometers at about 50% emission. One suitable europium chelate is N-(p-isothiocyanatobenzyl)-diethylene triamine tetraacetic acid-Eu⁺³.

In addition, lanthanide chelates that are inert, stable, and intrinsically fluorescent in aqueous solutions or suspensions may also be used in the present invention to negate the need for micelle-forming reagents, which are often used to protect chelates having limited solubility and quenching problems in aqueous solutions or suspensions. One example of such a chelate is 4-[2-(4-isothiocyanatophenyl)ethynyl]-2,6-bis([N,N-bis(carboxymethyl)amino]methyl)-pyridine [Ref: Lovgren, T., etal.; Clin. Chem. 42, 1196-1201 (1996)]. Several lanthanide chelates also show exceptionally high signal-to-noise ratios. For example, one such chelate is a tetradentate β-diketonate-europium chelate [Ref: Yuan, J. and Matsumoto, K.; Anal. Chem. 70, 596-601 (1998)]. In addition to the fluorescent labels described above, other labels that are suitable for use in the present invention may be described in U.S. Pat. No. 6,030,840 to Mullinax, et al.; U.S. Pat. No. 5,585,279 to Davidson; U.S. Pat. No. 5,573,909 to Singer, et al.; U.S. Pat. No. 6,242,268 to Wieder, et al.; and U.S. Pat. No. 5,637,509 to Hemmila, et al., which are incorporated herein in their entirety by reference thereto for all purposes.

Optically detectable substances, such as described above, may be used alone or in conjunction with a particle (sometimes referred to as “beads”). For instance, naturally occurring particles, such as nuclei, mycoplasma, plasmids, plastids, mammalian cells (e.g., erythrocyte ghosts), unicellular microorganisms (e.g., bacteria), polysaccharides (e.g., agarose), etc., may be used. Further, synthetic particles may also be utilized. For example, in one embodiment, latex particles that are labeled with a fluorescent dye are utilized. Although any synthetic particle may be used in the present invention, the particles are typically formed from polystyrene, butadiene styrenes, styreneacrylic-vinyl terpolymer, polymethylmethacrylate, polyethylmethacrylate, styrene-maleic anhydride copolymer, polyvinyl acetate, polyvinylpyridine, polydivinylbenzene, polybutyleneterephthalate, acrylonitrile, vinylchloride-acrylates, and so forth, or an aldehyde, carboxyl, amino, hydroxyl, or hydrazide derivative thereof. Other suitable particles may be described in U.S. Pat. No. 5,670,381 to Jou, et al. and U.S. Pat. No. 5,252,459 to Tarcha, et al. Commercially available examples of suitable fluorescent particles include fluorescent carboxylated microspheres sold by Molecular Probes, Inc. under the trade names “FluoSphere” (Red 580/605) and “TransfluoSphere” (543/620), as well as “Texas Red” and 5- and 6-carboxytetramethylrhodamine, which are also sold by Molecular Probes, Inc. In addition, commercially available examples of suitable colored, latex microparticles include carboxylated latex beads sold by Bang's Laboratory, Inc.

When utilized, the shape of the particles may generally vary. In one particular embodiment, for instance, the particles are spherical in shape. However, it should be understood that other shapes are also contemplated by the present invention, such as plates, rods, discs, bars, tubes, irregular shapes, etc. In addition, the size of the particles may also vary. For instance, the average size (e.g., diameter) of the particles may range from about 0.1 nanometers to about 1,000 microns, in some embodiments, from about 0.1 nanometers to about 100 microns, and in some embodiments, from about 1 nanometer to about 10 microns. For instance, “micron-scale” particles are often desired. When utilized, such “micron-scale” particles may have an average size of from about 1 micron to about 1,000 microns, in some embodiments from about 1 micron to about 100 microns, and in some embodiments, from about 1 micron to about 10 microns. Likewise, “nano-scale” particles may also be utilized. Such “nano-scale” particles may have an average size of from about 0.1 to about 10 nanometers, in some embodiments from about 0.1 to about 5 nanometers, and in some embodiments, from about 1 to about 5 nanometers.

In some instances, it may be desired to modify the detection probes in some manner so that they are more readily able to bind to the analyte or other substances. In such instances, the detection probes may be modified with certain specific binding members that are adhered thereto to form conjugated probes. Specific binding members generally refer to a member of a specific binding pair, i.e., two different molecules where one of the molecules chemically and/or physically binds to the second molecule. For instance, immunoreactive specific binding members may include antigens, haptens, aptamers, antibodies (primary or secondary), and complexes thereof, including those formed by recombinant DNA methods or peptide synthesis. An antibody may be a monoclonal or polyclonal antibody, a recombinant protein or a mixture(s) or fragment(s) thereof, as well as a mixture of an antibody and other specific binding members. The details of the preparation of such antibodies and their suitability for use as specific binding members are well known to those skilled in the art. Other common specific binding pairs include but are not limited to, biotin and avidin (or derivatives thereof), biotin and streptavidin, carbohydrates and lectins, complementary nucleotide sequences (including probe and capture nucleic acid sequences used in DNA hybridization assays to detect a target nucleic acid sequence), complementary peptide sequences including those formed by recombinant methods, effector and receptor molecules, hormone and hormone binding protein, enzyme cofactors and enzymes, enzyme inhibitors and enzymes, and so forth. Furthermore, specific binding pairs may include members that are analogs of the original specific binding member. For example, a derivative or fragment of the analyte, i.e., an analyte-analog, may be used so long as it has at least one epitope in common with the analyte.

The specific binding members may generally be attached to the detection probes using any of a variety of well-known techniques. For instance, covalent attachment of the specific binding members to the detection probes (e.g., particles) may be accomplished using carboxylic, amino, aldehyde, bromoacetyl, iodoacetyl, thiol, epoxy and other reactive or linking functional groups, as well as residual free radicals and radical cations, through which a protein coupling reaction may be accomplished. A surface functional group may also be incorporated as a functionalized co-monomer because the surface of the detection probe may contain a relatively high surface concentration of polar groups. In addition, although detection probes are often functionalized after synthesis, such as with poly(thiophenol), the detection probes may be capable of direct covalent linking with a protein without the need for further modification. For example, in one embodiment, the first step of conjugation is activation of carboxylic groups on the probe surface using carbodiimide. In the second step, the activated carboxylic acid groups are reacted with an amino group of an antibody to form an amide bond. The activation and/or antibody coupling may occur in a buffer, such as phosphate-buffered saline (PBS) (e.g., pH of 7.2) or 2-(N-morpholino) ethane sulfonic acid (MES) (e.g., pH of 5.3). The resulting detection probes may then be contacted with ethanolamine, for instance, to block any remaining activated sites. Overall, this process forms a conjugated detection probe, where the antibody is covalently attached to the probe. Besides covalent bonding, other attachment techniques, such as physical adsorption, may also be utilized in the present invention.

Referring again to FIG. 1, the chromatographic medium 23 also defines a detection zone 31 within which is immobilized a receptive material that is capable of binding to the conjugated detection probes. For example, in some embodiments, the receptive material may be a biological receptive material. Such biological receptive materials are well known in the art and may include, but are not limited to, antigens, haptens, protein A or G, neutravidin, avidin, streptavidin, captavidin, primary or secondary antibodies (e.g., polyclonal, monoclonal, etc.), and complexes thereof. In many cases, it is desired that these biological receptive materials are capable of binding to a specific binding member (e.g., antibody) present on the detection probes. The receptive material serves as a stationary binding site for complexes formed between the analyte and conjugated detection probes. Specifically, analytes, such as antibodies, antigens, etc., typically have two or more binding sites (e.g., epitopes). Upon reaching the detection zone 31, one of these binding sites is occupied by the specific binding member of the conjugated probe. However, the free binding site of the analyte may bind to the immobilized receptive material. Upon being bound to the immobilized receptive material, the complexed probes form a new ternary sandwich complex.

The detection zone 31 may generally provide any number of distinct detection regions so that a user may better determine the concentration of a particular analyte within a test sample. Each region may contain the same receptive materials, or may contain different receptive materials for capturing multiple analytes. For example, the detection zone 31 may include two or more distinct detection regions (e.g., lines, dots, etc.). The detection regions may be disposed in the form of lines in a direction that is substantially perpendicular to the flow of the test sample through the assay device 20. Likewise, in some embodiments, the detection regions may be disposed in the form of lines in a direction that is substantially parallel to the flow of the test sample through the assay device 20.

Although the detection zone 31 provides accurate results for detecting an analyte, it is sometimes difficult to determine the relative concentration of the analyte within the test sample under actual test conditions. Thus, the assay device 20 may also include a calibration zone 32. In this embodiment, the calibration zone 32 is positioned downstream from the detection zone 31. Alternatively, however, the calibration zone 32 may also be positioned upstream from the detection zone 31. The calibration zone 32 may be provided with a receptive material that is capable of binding to calibration probes or uncomplexed detection probes that pass through the length of the chromatographic medium 23. When utilized, the calibration probes may be formed from the same or different materials as the detection probes. Generally speaking, the calibration probes are selected in such a manner that they do not bind to the receptive material at the detection zone 31.

The receptive material of the calibration zone 32 may be the same or different than the receptive material used in the detection zone 31. For example, in one embodiment, the receptive material is a biological receptive material. In addition, it may also be desired to utilize various non-biological materials for the receptive material of the calibration zone 32. The polyelectrolytes may have a net positive or negative charge, as well as a net charge that is generally neutral. For instance, some suitable examples of polyelectrolytes having a net positive charge include, but are not limited to, polylysine (commercially available from Sigma-Aldrich Chemical Co., Inc. of St. Louis, Mo.), polyethylenimine; epichlorohydrin-functionalized polyamines and/or polyamidoamines, such as poly(dimethylamine-co-epichlorohydrin); polydiallyidimethyl-ammonium chloride; cationic cellulose derivatives, such as cellulose copolymers or cellulose derivatives grafted with a quaternary ammonium water-soluble monomer; and so forth. In one particular embodiment, CelQuat® SC-230M or H-100 (available from National Starch & Chemical, Inc.), which are cellulosic derivatives containing a quaternary ammonium water-soluble monomer, may be utilized. Moreover; some suitable examples of polyelectrolytes having a net negative charge include, but are not limited to, polyacrylic acids, such as poly(ethylene-co-methacrylic acid, sodium salt), and so forth. It should also be understood that other polyelectrolytes may also be utilized in the present invention, such as amphiphilic polyelectrolytes (i.e., having polar and non-polar portions). For instance, some examples of suitable amphiphilic polyelectrolytes include, but are not limited to, poly(styryl-b-N-methyl 2-vinyl pyridinium iodide) and poly(styryl-b-acrylic acid), both of which are available from Polymer Source, Inc. of Dorval, Canada. Further examples of internal calibration systems that utilize polyelectrolytes are described in more detail in U.S. patent app. Publication No. 2003/0124739 to Song, et al., which is incorporated herein in it entirety by reference thereto for all purposes.

In some cases, the chromatographic medium 23 may also define a control zone (not shown) that gives a signal to the user that the assay is performing properly. For instance, the control zone (not shown) may contain an immobilized receptive material that is generally capable of forming a chemical and/or physical bond with probes or with the receptive material immobilized on the probes. Some examples of such receptive materials include, but are not limited to, antigens, haptens, antibodies, protein A or G, avidin, streptavidin, secondary antibodies, and complexes thereof. In addition, it may also be desired to utilize various non-biological materials for the control zone receptive material. For instance, in some embodiments, the control zone receptive material may also include a polyelectrolyte, such as described above, that may bind to uncaptured probes. Because the receptive material at the control zone is only specific for probes, a signal forms regardless of whether the analyte is present. The control zone may be positioned at any location along the medium 23, but is typically positioned upstream from the detection zone 31.

Various formats may be used to test for the presence or absence of an analyte using the assay device 20. For instance, a “sandwich” format typically involves mixing the test sample with detection probes conjugated with a specific binding member (e.g., antibody) for the analyte to form complexes between the analyte and the conjugated probes. These complexes are then allowed to contact a receptive material (e.g., antibodies) immobilized within the detection zone. Binding occurs between the analyte/probe conjugate complexes and the immobilized receptive material, thereby localizing “sandwich” complexes that are detectable to indicate the presence of the analyte. This technique may be used to obtain quantitative or semi-quantitative results. Some examples of such sandwich-type assays are described by U.S. Pat. No. 4,168,146 to Grubb, et al. and U.S. Pat. No. 4,366,241 to Tom, et al., which are incorporated herein in their entirety by reference thereto for all purposes. In a competitive assay, the labeled probe is generally conjugated with a molecule that is identical to, or an analog of, the analyte. Thus, the labeled probe competes with the analyte of interest for the available receptive material. Competitive assays are typically used for detection of analytes such as haptens, each hapten being monovalent and capable of binding only one antibody molecule. Examples of competitive immunoassay devices are described in U.S. Pat. No. 4,235,601 to Deutsch, et al., U.S. Pat. No. 4,442,204 to Liotta, and U.S. Pat. No. 5,208,535 to Buechler, et al., which are incorporated herein in their entirety by reference thereto for all purposes. Various other device configurations and/or assay formats are also described in U.S. Pat. No. 5,395,754 to Lambotte, et al.; U.S. Pat. No. 5,670,381 to Jou, et al.; and U.S. Pat. No. 6,194,220 to Malick, et al., which are incorporated herein in their entirety by reference thereto for all purposes.

The actual configuration and structure of the optical reader used with the assay device 20 may generally vary as is readily understood by those skilled in the art. For example, optical detection techniques that may be utilized include, but are not limited to, luminescence (e.g., fluorescence, phosphorescence, etc.), absorbance (e.g., fluorescent or non-fluorescent), diffraction, etc. Typically, the optical reader is capable of emitting light and also registering a detection signal (e.g., transmitted or reflected light, emitted fluorescence or phosphorescence, etc.). For example, in one embodiment, a reflectance spectrophotometer may be utilized to detect the presence of probes that exhibit a visual color (e.g. dyed latex microparticles). One suitable reflectance spectrophotometer is described, for instance, in U.S. patent app. Pub. No. 2003/0119202 to Kaylor, et al., which is incorporated herein in its entirety by reference thereto for all purposes. In another embodiment, a reflectance-mode spectrofluorometer may be used to detect the presence of probes that exhibit fluorescence. Suitable spectrofluorometers and related detection techniques are described, for instance, in U.S. patent app. Pub. No. 2004/0043502 to Song, et al., which is incorporated herein in its entirety by reference thereto for all purposes. Likewise, a transmission-mode detection system may also be used to detect the presence of detection probes. Examples of such transmission-mode techniques are described in more detail in co-owned, co-pending U.S. patent application entitled “Transmission-Based Luminescent Detection Systems” (filed on Dec. 22, 2004; Express Mail Receipt No. 599453864) and co-owned, co-pending U.S. patent application entitled “Transmission-Based Optical Detection Systems” (filed on Dec. 22, 2004; Express Mail Receipt No. 599453855), both of which are incorporated herein in their entirety by reference thereto for all purposes.

Referring again to FIG. 1, for example, the illustrated detection system employs an optical reader 50 that contains an illumination source 52 and a detector 54. As shown, the detector 54 is positioned adjacent to the support 21 and the illumination source 52 is positioned adjacent to the second surface 14 of the chromatographic medium 23. Likewise, the detector 54 may be positioned adjacent to the second surface 14 of the chromatographic medium 23 and the illumination source 52 may be positioned adjacent to the support 21. Thus, the illumination source 52 may emit light simultaneously onto the detection and calibration zones 31 and 32, and the detector 54 may likewise also simultaneously receive a luminescent signal from the excited probes at the detection and calibration zones 31 and 32. Alternatively, the illumination source 52 may be constructed to successively emit light onto the detection zone 31 and the calibration zone 32. In addition, a separate illumination source and/or detector (not shown) may also be used for the calibration zone 32.

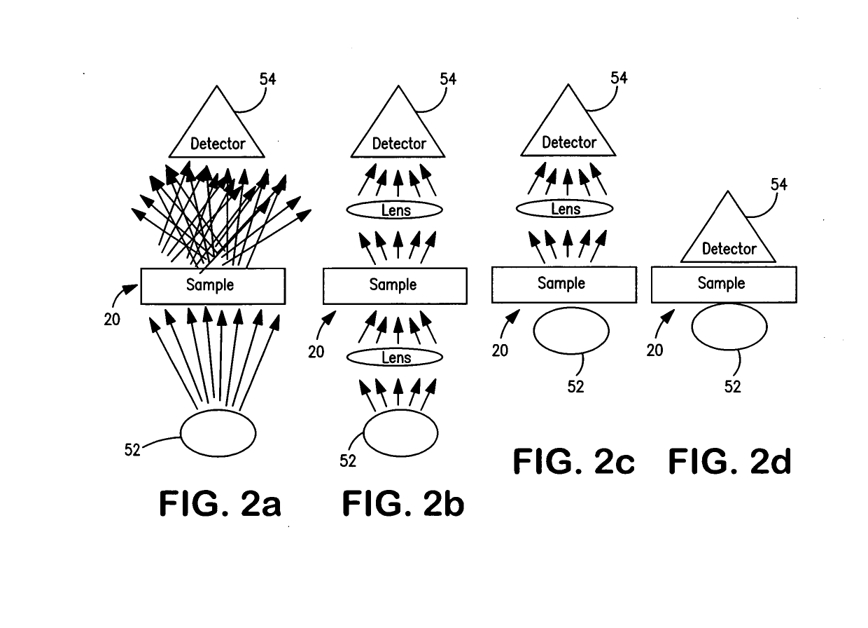

To improve the signal-to-noise ratio of the optical detection system without the need for certain types of complex and expensive optical components, such as lenses or other light guiding elements, the distance of the illumination source 52 and/or detector 54 from the assay device 20 may be minimized in some embodiments. For instance, as shown in FIG. 2a, light (indicated by directional arrows) traveling a relatively large distance tends to diffuse, thereby causing some photons to miss the test sample or the detector 54. To reduce light scattering, lenses may be employed to focus the light in the desired direction, such as shown in FIG. 2b. However, as shown in FIGS. 2c and 2d, the need for such expensive and complex equipment may be reduced by simply moving the illumination source 52 and/or detector 54 closer to the assay device 20. The use of a shorter light path results in less diffusion of the light. For example, FIG. 2c illustrates an embodiment in which the illumination source 52 is positioned closer to the assay device 20, and FIG. 2d illustrates an embodiment in which both the illumination source 52 and detector 54 are positioned closer to the assay device 20. Thus, in some embodiments, the illumination source 52 and/or detector 54 may be positioned less than about 5 millimeters, in some embodiments less than about 3 millimeters, and in some embodiments, less than about 2 millimeters from the assay device 20. In some cases, it may be desired to keep the illumination source 52 and/or detector 54 at a distance that is large enough to avoid contamination of any biological reagents. For example, the illumination source 52 and/or detector 54 may sometimes be positioned at a distance of from about 1 to about 3 millimeters from the assay device 20.

Generally speaking, the illumination source 52 may be any device known in the art that is capable of providing electromagnetic radiation at a sufficient intensity to cause probes to produce a detection signal. The electromagnetic radiation may include light in the visible or near-visible range, such as infrared or ultraviolet light. For example, suitable illumination sources that may be used in the present invention include, but are not limited to, light emitting diodes (LED), flashlamps, cold-cathode fluorescent lamps, electroluminescent lamps, and so forth. The illumination may be multiplexed and/or collimated. In some cases, the illumination may be pulsed to reduce any background interference. Further, illumination may be continuous or may combine continuous wave (CW) and pulsed illumination where multiple illumination beams are multiplexed (e.g., a pulsed beam is multiplexed with a CW beam), permitting signal discrimination between a signal induced by the CW source and a signal induced by the pulsed source. For example, in some embodiments, LEDs (e.g., aluminum gallium arsenide red diodes, gallium phosphide green diodes, gallium arsenide phosphide green diodes, or indium gallium nitride violet/blue/ultraviolet (UV) diodes) are used as the pulsed illumination source 52. One commercially available example of a suitable UV LED excitation diode suitable for use in the present invention is Model NSHU55OE (Nichia Corporation), which emits 750 to 1000 microwatts of optical power at a forward current of 10 milliamps (3.5-3.9 volts) into a beam with a full-width at half maximum of 10 degrees, a peak wavelength of 370-375 nanometers, and a spectral half-width of 12 nanometers.

In some cases, the illumination source 52 may provide diffuse illumination to the assay device 20. In this manner, the reliance on certain external optical components, such as diffusers, may be virtually eliminated. For example, in some embodiments, an array of multiple point light sources (e.g., LEDs) may simply be employed to provide relatively diffuse illumination to the device 20. Another particularly desired illumination source that is capable of providing diffuse illumination in a relatively inexpensive manner is an electroluminescent (EL) device. An EL device is generally a capacitor structure that utilizes a luminescent material (e.g., phosphor particles) sandwiched between electrodes, at least one of which is transparent to allow light to escape. Application of a voltage across the electrodes generates a changing electric field within the luminescent material that causes it to emit light. Examples of such EL devices are described in more detail in co-owned, co-pending U.S. patent application entitled “Electroluminescent Illumination Source for Optical Detection Systems” (filed on Dec. 22, 2004; Express Mail Receipt No. 599453847), which is incorporated herein in its entirety by reference thereto for all purposes.

The detector 54 may generally be any device known in the art that is capable of sensing an optical signal. For instance, the detector 54 may be an electronic imaging detector that is configured for spatial discrimination. Some examples of such electronic imaging sensors include high speed, linear charge-coupled devices (CCD), charge-injection devices (CID), complementary-metal-oxide-semiconductor (CMOS) devices, and so forth. Such image detectors, for instance, are generally two-dimensional arrays of electronic light sensors, although linear imaging detectors (e.g., linear CCD detectors) that include a single line of detector pixels or light sensors, such as, for example, those used for scanning images, may also be used. Each array includes a set of known, unique positions that may be referred to as “addresses.” Each address in an image detector is occupied by a sensor that covers an area (e.g., an area typically shaped as a box or a rectangle). This area is generally referred to as a “pixel” or pixel area. A detector pixel, for instance, may be a CCD, CID, or a CMOS sensor, or any other device or sensor that detects or measures light. The size of detector pixels may vary widely, and may in some cases have a diameter or length as low as 0.2 micrometers.

In other embodiments, the detector 54 may be a light sensor that lacks spatial discrimination capabilities. For instance, examples of such light sensors may include photomultiplier devices, photodiodes, such as avalanche photodiodes or silicon photodiodes, and so forth. Silicon photodiodes are sometimes advantageous in that they are inexpensive, sensitive, capable of high-speed operation (short risetime / high bandwidth), and easily integrated into most other semiconductor technology and monolithic circuitry. In addition, silicon photodiodes are physically small, which enables them to be readily incorporated into a system for use with a membrane-based device. If silicon photodiodes are used, then the wavelength range of the emitted signal may be within their range of sensitivity, which is 400 to 1100 nanometers.

Referring again to FIG. 1, the optical properties of the support 21 may be selectively controlled to optimize the performance of the optical detection system, particularly the illumination source 52 and the detector 54. For example, in one particular embodiment, the support 21 is optically transmissive to allow light to travel from the illumination source 52 to the detector 54. In addition, the support 21 may function as an optical filter of the detection system. Thus, in the illustrated embodiment, light from the illumination source 52 is absorbed by probes (not shown) present at the detection zone 31 and/or calibration zone 32. The probes produce a signal that is attenuated by the optical filter before reaching the detector 54. The optical filter may be particularly useful in luminescent detection system and have, for example, have high transmissibility in a desired wavelength range(s) and low transmissibility in one or more undesirable wavelength band(s) to filter out undesirable wavelengths from the detector 54. The optical detection system may also include an additional optical filter (not shown) positioned between the illumination source 52 and the chromatographic medium 23. This additional optical filter may have high transmissibility in the excitation wavelength range(s) and low transmissibility in one or more undesirable wavelength band(s). Alternatively, an additional optical filter may be integrated into the illumination source 52 and/or detector 54. In other embodiments, the support 21 may contain a mask, light guiding element, lens, diffuser, etc. For example, the support 21 may be a light diffuser formed from a polymeric film containing optically functional diffusing elements, such as “white” titanium dioxide particles. This may be particularly desired for optical detection systems that employ “point” light sources, such as LEDs.

Generally speaking, qualitative, quantitative, or semi-quantitative determination of the presence or concentration of an analyte may be achieved in accordance with the present invention. For example, in one embodiment, the amount of the analyte may be quantitatively or semi-quantitatively determined by correlating the intensity of the signal, Iₛ, of the probes captured at the detection zone 31 with a predetermined analyte concentration. In some embodiments, the intensity of the signal, Iₛ, may also be compared with the intensity of the signal, I_{c}, of the probes captured at the calibration zone 32. The intensity of the signal, Iₛ, may be compared to the intensity of the signal, I_{c}. In this embodiment, the total amount of the probes at the calibration zone 32 is predetermined and known and thus may be used for calibration purposes. For example, in some embodiments (e.g., sandwich assays), the amount of analyte is directly proportional to the ratio of Iₛ to I_{c}. In other embodiments (e.g., competitive assays), the amount of analyte is inversely proportional to the ratio of Iₛ to I_{c}. Based upon the intensity range in which the detection zone 31 falls, the general concentration range for the analyte may be determined. As a result, calibration and sample testing may be conducted under approximately the same conditions at the same time, thus providing reliable quantitative or semi-quantitative results, with increased sensitivity.

If desired, the ratio of Iₛ to I_{c} may be plotted versus the analyte concentration for a range of known analyte concentrations to generate a calibration curve. To determine the quantity of analyte in an unknown test sample, the signal ratio may then be converted to analyte concentration according to the calibration curve. It should be noted that alternative mathematical relationships between Iₛ and I_{c} may be plotted versus the analyte concentration to generate the calibration curve. For example, in one embodiment, the value of Iₛ/(Iₛ+I_{c}) may be plotted versus analyte concentration to generate the calibration curve.

A microprocessor may optionally be employed to convert the measurement from the detector 54 to a result that quantitatively or semi-quantitatively indicates the presence or concentration of the analyte. The microprocessor may include memory capability to allow the user to recall the last several results. Those skilled in the art will appreciate that any suitable computer-readable memory devices, such as RAM, ROM, EPROM, EEPROM, flash memory cards, digital video disks, Bernoulli cartridges, and so forth, may be used in the present invention. Optical density (grayscale) standards may also be used to facilitate a quantitative result as is well known in the art. Further, any known software may optionally be employed for data collection. For example, Logitech camera software may be used to collect data obtained from a Logitech camera-based detector. After the images are saved, they may be analyzed using any known commercial software package, such as ImageQuant from Molecular Dynamics of Sunnyvale, Calif. If desired, the results may be conveyed to a user using a liquid crystal (LCD) or LED display.

While the invention has been described in detail with respect to the specific embodiments thereof, it will be appreciated that those skilled in the art, upon attaining an understanding of the foregoing, may readily conceive of alterations to, variations of, and equivalents to these embodiments. Accordingly, the scope of the present invention should be assessed as that of the appended claims and any equivalents thereto.

1. An optical detection system for detecting the presence or quantity of an analyte residing in a test sample, said system comprising:
an optical reader that comprises an illumination source and a detector, said illumination source being capable of providing electromagnetic radiation and said detector being capable of registering a detection signal; and
an assay device that includes a porous membrane having a first surface and an opposing second surface, said porous membrane being in communication with detection probes that are capable of producing said detection signal when contacted with said electromagnetic radiation, wherein said first surface of said porous membrane is carried by a support, said support having a thickness of from about 100 to about 5,000 micrometers, wherein said support is provided with an optically functional material that is selectively tailored to one or more optical properties of said optical reader.

2. The optical detection system of claim 1, wherein said optically functional material is an optical filter.

3. The optical detection system of claim 2, wherein said optical filter is a high-pass, low-pass, or band-pass filter.

4. The optical detection system of claim 3, wherein said optical filter has a high transmissibility at one or more wavelengths specific to said electromagnetic radiation.

5. The optical detection system of claim 3, wherein said optical filter has a high transmissibility at one or more wavelengths specific to said detection signal.

6. The optical detection system of claim 1, wherein said optically functional material is a light guide element.

7. The optical detection system of claim 7, wherein said light guide element is a micro-optic lens.

8. The optical detection system of claim 1, wherein said optically functional material is a mask.

9. The optical detection system of claim 1, wherein said optically functional material is a diffusing element and said support functions as a diffuser.

10. The optical detection system of claim 9, wherein said support functions as a holographic diffuser.

11. The optical detection system of claim 1, wherein said porous membrane defines a detection zone within said detection probes are capable of being immobilized, wherein the amount of the analyte within the test sample is proportional to the intensity of said detection signal generated within said detection zone.

12. The optical detection system of claim 1, wherein said illumination source is positioned adjacent to said support and said detector is positioned adjacent to said second surface of said porous membrane.

13. The optical detection system of claim 1, wherein said detector is positioned adjacent to said support and said illumination source is positioned adjacent to said second surface of said porous membrane.

14. The optical detection system of claim 1, wherein said detector and said illumination source are positioned adjacent to said second surface of said porous membrane.

15. The optical detection system of claim 1, further comprising an additional support provided with an optically functional material, said additional support being positioned adjacent to said second surface of said porous membrane.

16. The optical detection system of claim 1, wherein said support has a thickness of from about 150 to about 2,000 micrometers.

17. The optical detection system of claim 1, wherein said support has a thickness of from about 250 to about 1,000 micrometers.

18. The optical detection system of claim 1, wherein said optically functional material is applied to one or more surfaces of said support.

19. The optical detection system of claim 18, wherein the location of said optically functional material on said support corresponds to a detection zone defined by said porous membrane.

20. The optical detection system of claim 1, wherein the system further comprises an optically transparent adhesive.

21. The optical detection system of claim 20, wherein said adhesive laminates said support to said porous membrane.

22. An optical detection system for detecting the presence or quantity of an analyte residing in a test sample, said system comprising:
an optical reader that comprises an illumination source and a detector, said illumination source being capable of providing electromagnetic radiation and said detector being capable of registering a detection signal; and
an assay device that includes a porous membrane having a first surface and an opposing second surface, said porous membrane being in communication with detection probes that are capable of producing said detection signal when contacted with said electromagnetic radiation, wherein said illumination source and said detector are positioned on opposing sides of said assay device so that said porous membrane is positioned in the electromagnetic radiation path defined between said illumination source and said detector;
wherein said first surface of said porous membrane is carried by an optically transmissive support, said optically transmissive support having a thickness of from about 150 to about 2,000 micrometers, wherein said optically transmissive support is provided with an optically functional material that is selectively tailored to one or more optical properties of said optical reader.

23. The optical detection system of claim 22, wherein said optically functional material is an optical filter.

24. The optical detection system of claim 22, wherein said optically functional material is a diffusing element and said support functions as a diffuser.

25. The optical detection system of claim 22, wherein the system further comprises an optically transparent adhesive.

26. The optical detection system of claim 25, wherein said adhesive laminates said support to said porous membrane.

27. A method for detecting the presence or quantity of an analyte within a test sample, said method comprising:
providing an optical reader;
providing a chromatographic medium for an assay device;
selectively controlling the optical properties of a support for said chromatographic medium to correspond with one or more optical requirements of said optical reader, said support having a thickness of from about 100 to about 5,000 micrometers.

28. The method of claim 27, further comprising:
contacting the test sample with said chromatographic medium;
supplying electromagnetic radiation to said test sample to cause the production of a detection signal; and
registering said detection signal.

29. The method of claim 28, wherein said support has a high transmissibility at one or more wavelengths specific to said electromagnetic radiation.

30. The method of claim 28, wherein said support has a high transmissibility at one or more wavelengths specific to said detection signal.

31. The method of claim 28, wherein said support diffuses said electromagnetic radiation, said detection signal, or both.
